(19) United States
(12) Patent Application Publication
(10) Pub. No.: US 2026/0235610 A1
(43) Pub. Date: Aug. 13, 2026

(54) METHOD OF PREDICTING SUCCESS OF A CANCER THERAPY

(71) Applicant: IMMUSMOL SAS, Bordeaux (FR)

(72) Inventor: Alban BESSEDE, Bordeaux (FR)

(73) Assignee: IMMUSMOL SAS, Bordeaux (FR)

(21) Appl. No.: 19/153,480

(22) PCT Filed: Feb. 5, 2024
(86) PCT No.: PCT/EP2024/052783

§ 371 Date: Aug. 4, 2025

(30) Foreign Application Priority Data
Feb. 3, 2023 (EP) 23154976.7

(51) Int. Cl. G01N 33/575; A61K 31/506; A61P 35/00

(52) U.S. Cl. CPC G01N 33/5758; A61K 31/506; A61P 35/00; G01N 2333/90245; G01N 2333/90254; G01N 2333/978; G01N 2333/988

(57) ABSTRACT

The present invention relates to an in vitro method of determining the need of a patient being diagnosed for, suffering from or being at risk of developing cancer, for a given therapy, the method comprising determining, in vitro, in one or more samples collected from said patient, the activity of at least one enzyme selected from the group consisting of Tryptophan Hydroxylase, 5-HTP decarboxylase, Arginase and/or Nitric oxide synthase (NOS), and determining the patient to be in need of a combined treatment comprising anti-cancer therapy, and an inhibitor of Tryptophan Hydroxylase, 5-HTP decarboxylase, Arginase and/or Nitric oxide synthase (NOS) if a high activity of the respective enzyme is determined in step a), while determining the patient to be in need of a cancer treatment comprising anti-cancer therapy, if a low activity of the respective enzyme is determined in step a) (FIG. 5).

The present invention is related to a method of predicting success of a cancer therapy.

### BACKGROUND OF THE INVENTION

Cancer is still the world's most important health challenge. Despite progress being made in the development of chemotherapy, targeted therapy, anti-angiogenic therapy, tyrosine kinase inhibitors, and/or immunotherapy, too many patients still die eventually, due to insufficient responses.

### SUMMARY OF THE INVENTION

It is an object of the present invention to provide better methods of predicting success of a cancer therapy.

It is another object of the the present invention to establish new treatment options for cancer patients.

It is another object of the present invention to provide methods to identify cancer patients subgroups that may receive benefit of particular treatment modalities.

### EMBODIMENTS OF THE INVENTION

These and other objects are met with methods and means according to the independent claims of the present invention. The dependent claims are related to preferred embodiments. It is yet to be understood that value ranges delimited by numerical values are to be understood to include the said delimiting values.

### BRIEF DESCRIPTION OF THE FIGURES

FIG. 1 shows the tryptophan degradation pathway.

FIG. 2 shows the Urea cycle.

FIG. 3 shows an experimental protocol underlying example 1.

FIGS. 4-6 show Kaplan Meier curves as obtained after analysis of example 1.

FIGS. 7-9 show the enzyme activity (FIG. 7, Tryptophan Hydroxylase (TPH1); FIG. 8, Nitric Oxide Synthase (NOS); FIG. 9, Arginase) evaluated in plasma from human healthy subjects and patients suffering from different types of cancer.

FIG. 10 shows the experimental design used to evaluate the impact of TPH1^{High} conditioned culture media on immune cell activation.

FIG. 11 shows the conditioned media characteristics that were used to evaluate the immune cell activation.

FIG. 12 shows evaluation of a QGP1-conditioned media on PBMCs activation.

### SUMMARY OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

According to a first aspect of the invention, an in vitro method of determining the need of a patient being diagnosed for, suffering from or being at risk of developing cancer, for a given therapy is provided. The method comprises
a) determining, in vitro, in one or more samples collected from said patient, the activity of at least one enzyme selected from the group consisting of Tryptophan Hydroxylase, 5-HTP decarboxylase, Arginase and/or Nitric oxide synthase (NOS), and
b) determining the patient to be in need of a combined treatment comprising
   (i) anti-cancer therapy, and
   (ii) an inhibitor of Tryptophan Hydroxylase, 5-HTP decarboxylase, Arginase and/or Nitric oxide synthase (NOS)
if a high activity of the respective enzyme is determined in step a),
c) while determining the patient to be in need of a cancer treatment comprising anti-cancer therapy, if a low activity of the respective enzyme is determined in step a).

According to one embodiment, the activity of Tryptophan Hydroxylase is determined and/or the combined treatment comprises an inhibitor of Tryptophan Hydroxylase. According to one embodiment, the Tryptophan Hydroxylase is Tryptophan Hydroxylase 1 (TPH1).

According to one embodiment of the invention, the activity of Tryptophan Hydroxylase is determined by measuring the levels of at least a pair of metabolites selected from
a) Tryptophan (Trp) and 5-hydroxytryptophan (5-HTP), and/or
b) Tryptophan (Trp) and 5-hydroxytryptamine (5-HT).

According to one embodiment of the invention, the activity of 5-HTP decarboxylase is determined by measuring the levels of at least a pair of metabolites selected from
c) Tryptophan (Trp) and 5-hydroxytryptamine (5-HT), and/or
d) 5-hydroxythroptophan (5-HTP) and 5-hydroxytryptamine (5-HT).

According to one embodiment of the invention, the activity of Arginase is determined by measuring the levels of the metabolites Arginine (Arg) and Ornithine (Orn).

According to one embodiment of the invention, the activity of Nitric oxide synthase (NOS) is determined by measuring the levels of the metabolites Arginine (Arg) and Citrulline (Cit).

According to one embodiment of the invention, the activity of Tryptophan Hydroxylase is determined by calculating the ratio of the levels of
a) 5-hydroxythroptophan (5-HTP) and Tryptophan (Trp), and/or
b) 5-hydroxytryptamine (5-HT) and Tryptophan (Trp).

According to one embodiment of the invention, the activity of 5-HTP decarboxylase is determined by calculating the ratio of the levels of
c) 5-hydroxytryptamine (5-HT) and Tryptophan (Trp) and/or
d) 5-hydroxytryptamine (5-HT) and 5-hydroxythroptophan (5-HTP).

According to one embodiment of the invention, the activity of Arginase is determined by calculating the ratio of the levels of Ornithine (Orn) and Arginine (Arg), and/or the activity of Nitric oxide synthase (NOS) is determined by calculating the ratio of the levels of Citrulline (Cit) and Arginine (Arg).

The following table summarizes details of one embodiment according to which the enzyme activity is determined.

TABLE 1
Preferred ways of determining enzyme activity

| Activity of | Ratio of level of |
|---|---|
| Tryptophan Hydroxylase | [5-HTP/Trp] and/or [5-HT/Trp] |
| 5-HTP decarboxylase | [5-HT/Trp] and/or [5-HT/5-HTP] |
| Arginase | [Orn/Arg] |
| NOS | [Cit/Arg] |

In one embodiment, a high ratio of [5-HT/Trp] and/or [5-HTP/Trp] indicates a high activity of Tryptophan Hydroxylase. In one embodiment, a high ratio of [5-HT/Trp] and/or [5-HT/5-HTP]] indicates a high activity of 5-HTP decarboxylase. In one embodiment, a high ratio of [Orn/Arg] indicates a high activity of Arginase. In one embodiment, a high ratio of [Cit/Arg] indicates a high activity of Nitric oxide synthase.

In one embodiment, a low ratio of [5-HT/Trp] and/or [5-HTP/Trp] indicates a low activity of Tryptophan Hydroxylase. In one embodiment, a low ratio of [5-HT/Trp] and/or [5-HT/5-HTP]] indicates a low activity of 5-HTP decarboxylase. In one embodiment, a low ratio of [Orn/Arg] indicates a low activity of Arginase. In one embodiment, a low ratio of [Cit/Arg]] indicates a low activity of Nitric oxide synthase.

In one embodiment, the ratio of two metabolites will be classified as “high” if it is increased in comparison to at least one respective ratio determined in a sample collected from a patient not being diagnosed for, suffering from or being at risk of developing cancer. In one embodiment, the ratio of two metabolites will be classified as “low” if it is reduced in comparison to at least one respective ratio determined in a sample collected from a patient not being diagnosed for, suffering from or being at risk of developing cancer.

In one embodiment, the ratio of two metabolites will be classified as “high” if it is higher than a given threshold, examples of which are provided herein. In one embodiment, the ratio of two metabolites will be classified as “low” if it is lower than a given threshold, examples of which are provided herein.

In such way, determining the ratios of these metabolites serves as a surrogate for determining the activity of the respective enzymes per se.

In one embodiment, the activity of a respective enzyme will be classified as “low” if it is reduced in comparison to at least one respective activity in a sample collected from a patient not being diagnosed for, suffering from or being at risk of developing cancer. In one embodiment, the activity of a respective enzyme will be classified as “high” if it is increased in comparison to at least one respective activity in a sample collected from a patient not being diagnosed for, suffering from or being at risk of developing cancer.

In one embodiment, the activity of a respective enzyme will be classified as “low” if it is below a given threshold as disclosed herein. In one embodiment, the activity of a respective enzyme will be classified as “high” if it is above a given threshold as disclosed herein.

As used herein, the term “level of a molecule or metabolite” refers, in one embodiment, to the concentration or titer of said molecule or metabolite in a sample. In that case, the term relates to an absolute value, like e.g., μg/mL, mg/mL, μmol/L or the like.

As used herein, the term “metabolite” refers to an educt or product of an amino acid in a given metabolic pathway. An educt, in this context, is an upstream metabolite, whereas a product is a downstream metabolite. Serotonine (5-HT) is for example a product, or downstream metabolite, of Tryptophan (Trp), while Tryptophan (Trp) is an educt, or upstream metabolite, of 5-Hydroxytryptophan (5-HTP). Citrulline (Cit) and Ornithine are downstream metabolites of Arginine (Arg).

As used herein, the term “ratio of the levels of two or more molecules or metabolites” refers, in one embodiment, to the arithmetic quotient of the concentrations or titers of said molecules or metabolites, preferably in the same sample. For example, if metabolite A has a concentration of 5 μg/mL, and metabolite B has a concentration of 3 μg/mL, the ratio [A/B] is 1.67, while the ratio or quotient [B/A] is 0.6. It should be noted that, contrary to the level, titer or concentration of a molecule or metabolite, the ratio or quotient of two molecules or metabolites is dimensionless.

As used herein, the determination of levels of molecules encompasses a variety of methodologies, parameters and threshold levels, as will be discussed hereinbelow.

As will be described elsewhere herein, inhibitors of Tryptophan Hydroxylase, 5-HTP decarboxylase, Arginase and/or Nitric oxide synthase are known in the art. However, they have so far been described for different therapeutical applications other than oncology only, like e.g., nausea, depression, or cardiovascular diseases.

The following table shows exemplary thresholds for determining the enzyme activity

TABLE 2
exemplary thresholds for determining the enzyme activity

| metabolite | | threshold | threshold | | If value below | If value above |
|---|---|---|---|---|---|---|
| or ratio | parameter | range | embodiment | | threshold | threshold |
| Arg | level/concentration | 34-62 μM | 42 | μM | Activity of | |
| | | | | | Arginase or | |
| | | | | | NOS high | |
| Trp | level/concentration | 35-60 μM | 34 | μM | Activity of | |
| | | | | | Tryptophan | |
| | | | | | hydroxylase | |
| | | | | | high | |
| 5-HT | level/concentration | 15-40 nM | | | — | Activity of |
| | | | | | | Tryptophan |
| | | | | | | hydroxylase |
| | | | | | | or 5HTP |
| | | | | | | decarboxylase |
| | | | | | | high |
| 5-HTP | level/concentration | 4-15 nM | 5 | nM | | Activity of |

| | | | | | |
|---|---|---|---|---|---|
| | | | | | Tryptophan |
| | | | | | hydroxylase |
| | | | | | high |
| Orn | level/concentration | 60-90 μM | | Activity of | |
| | | | | Arginase high | |
| Cit | level/concentration | 10-40 μM | | Activity of | |
| | | | | NOS high | |
| [5-HT/Trp] | ratio/quotient | 10-300 | 14 | | Activity of |
| (nM/μM) | (dimensionless) | | | | Tryptophan |
| | | | | | hydroxylase |
| | | | | | or 5HTP |
| | | | | | decarboxylase |
| | | | | | high |
| [5- | ratio/quotient | 0.10-0.30 | 0.14 | | Activity of |
| HTP/Trp] | (dimensionless) | | | | Tryptophan |
| (nM/μM) | | | | | hydroxylase |
| | | | | | high |
| [5-HT/5- | ratio/quotient | 1.5-10 | | | Activity |
| HTP] | (dimensionless) | | | | of 5HTP |
| (nM/nM) | | | | | decarboxylase |
| | | | | | high |
| [Orn/Arg] | ratio/quotient | 0.5-2 | 1.2 | | Activity of |
| (μM/μM) | (dimensionless) | | | | Arginase high |
| [Cit/Arg] | ratio/quotient | 0.3-0.6 | 0.4 | | Activity of |
| (μM/μM) | (dimensionless) | | | | NOS high |

As can be seen, the absolute levels/concentrations of the metabolites can be determined only for determining the respective enzyme activity. Likewise, the ratio of levels/concentrations of two metabolites can be determined for determining the respective enzyme activity.

In one embodiment, it is preferred to determine the ratio of levels/concentrations of two metabolites for determining the respective enzyme activity.

In a specific embodiment thereof, it is preferred to determine [5-HT/Trp] to determine the activity of Tryptophan hydroxylase or 5HTP decarboxylase.

In a another specific embodiment thereof, it is preferred to determine [5-HTP/Trp] to determine the activity of Tryptophan hydroxylase.

In another specific embodiment thereof, it is preferred to determine [5-HT/5-HTP] to determine the activity of 5HTP decarboxylase.

In another specific embodiment thereof, it is preferred to determine [Orn/Arg] to determine the activity of arginase.

In another specific embodiment thereof, it is preferred to determine [Cit/Arg] to determine the activity of NOS.

In one embodiment, it is preferred to determine the levels/concentrations of one or more metabolites for determining the respective enzyme activity.

In a specific embodiment thereof, it is preferred to determine the levels/concentration of Arginine to determine the activity of Arginase or NOS.

In a another specific embodiment thereof, it is preferred to determine the levels/concentration of Tryoptophan to determine the activity of Tryptophan hydroxylase.

According to yet another embodiment of the invention, the activity of at least one of
Tryptophan Hydroxylase
5-HTP decarboxylase
Arginase
Nitric oxide synthase (NOS)
   is determined by measuring the expression level thereof in one or samples collected from the patient. Such samples can be, for example, tumor samples obtained from a biopsy, or circulating cells (like e.g. B-cells).

Ways of determining the expression level of such enzyme, include, but are not limited, to
RNAseq (see e.g. Corchete et al 2020, the content of which is incorporated herein in its entirety for enablement purposes)
RT-PCR/qPCR (see e.g. Holland et al. 1991, the content of which is incorporated herein in its entirety for enablement purposes)
Immunohistochemistry (see e.g. Magaki et al 2019, the content of which is incorporated herein in its entirety for enablement purposes) and/or
Flow cytometry for circulating immune cells (see e.g. Mckinnon 2018 and Gaynor 1996, the content of which is incorporated herein in its entirety for enablement purposes)

These methods are well known in the art and fully within the routine of the skilled artisan. The present disclosure provides all information (e.g., sequence references) related to the enzymes of interest to enable the skilled artisan to produce, e.g., labelling antibodies (for e.g. Immunohistochemistry or Immunohistochemistry based Flow cytometry) or probes and primers (for e.g. TaqMan based RT-PCR, or RT-PCR based Flow cytometry).

In case the enzyme activity has been determined to be high, the patient is determined to be in need of a combined treatment comprising anti-cancer therapy, and an inhibitor of Tryptophan Hydroxylase, 5-HTP decarboxylase, Arginase and/or Nitric oxide synthase (NOS), respectively. In case the enzyme activity has been determined to be low, the patient is determined to be in need of a of a cancer treatment comprising anti-cancer therapy

According to one embodiment, the activity of Tryptophan Hydroxylase is determined and/or the combined treatment comprises an inhibitor of Tryptophan Hydroxylase. According to one embodiment, the Tryptophan Hydroxylase is Tryptophan Hydroxylase 1 (TPH1).

According to one embodiment of the invention, at least one metabolite is in L-configuration.

According to one embodiment of the invention, said one or more samples from the patient have been collected before treatment onset.

According to embodiments of the invention, said one or more samples from the patient have been derived from at least one selected from the group consisting of
plasma sample,
serum sample,
urine sample,
saliva sample,
liquor sample, and/or
whole blood sample.

According to embodiments of the invention, the level of at least one metabolite is determined by at least one method selected from the group consisting of
Immunoassay
Gas Chromatography/Mass Spectroscopy (GC/MS)
High Performance Liquid Chromatography (HPLC), and/or
Liquid Chromatography/Mass spectroscopy (LC/MS).

Immunoassays require relatively non-sophisticated infrastructure and can be used in point of care locations or small diagnostic laboratories.

Gas chromatography and mass spectrometry have higher demands regarding infrastructure and, as such, can be used in larger clinical centres and research institutions.

According to embodiments of the invention, the anti-cancer therapy comprises at least one selected from the group consisting of
chemotherapy
targeted therapy
administration of one or more anti-angiogenic drugs,
administration of one or more TKI (tyrosine kinase inhibitors), and/or
an immunotherapeutic approach.

The term “chemotherapy”, as used herein, refers to a cancer treatment method wherein one or more anti-cancer drugs are administered to a patient, this anti-cancer drug affecting cell growth and cell division. Chemotherapy in general affects replication of both cancerous cells and normal cells

Chemotherapeutic agents encompass, inter alia, Alkylating agents, Antimetabolites, Anti-microtubule agents, Topoisomerase inhibitors, Cytotoxic antibiotics

Alkylating agents are so named because of their ability to alkylate many molecules, including proteins, RNA and DNA. The subtypes of alkylating agents are the nitrogen mustards, nitrosoureas, tetrazines, aziridines, cisplatins and derivatives, and non-classical alkylating agents.

Antimetabolites are a group of molecules that impede DNA and RNA synthesis. Many of them have a similar structure to the building blocks of DNA and RNA. Subtypes of the antimetabolites are the anti-folates, fluoropyrimidines, deoxynucleoside analogues and thiopurines.

Anti-microtubule agents are chemicals that block cell division by preventing microtubule function. Vinca alkaloids and taxanes are the two main groups of anti-microtubule agents.

Topoisomerase inhibitors are drugs that affect the activity of the two enzymes topoisomerase I and topoisomerase II. Two topoisomerase I inhibitors, irinotecan and topotecan, are semi-synthetically derived from camptothecin. Topoisomerase II inhibitors include etoposide, doxorubicin, mitoxantrone and teniposide, as well as novobiocin, merbarone, and aclarubicin,

The cytotoxic antibiotics are a varied group of drugs that have various mechanisms of action. The common theme that they share in their chemotherapy indication is that they interrupt cell division. The most important subgroup is the anthracyclines (with doxorubicin and daunorubicin) and the bleomycins; other prominent examples include mitomycin C and actinomycin.

As used herein, the term “targeted therapy” refers to a cancer treatment method comprising the administration of molecules that specifically block the growth of cancer cells by interfering with specific targeted molecules which are necessary for carcinogenesis and tumor growth.

Typical targeted therapies include anti Her-2 agents like Trastuzumab, Pertuzumab and Margetuximab (Margenza), Ado-trastuzumab emtansine, Fam-trastuzumab deruxtecan, endocrine agents like tamoxifen m CDK4/6 inhibitors like palbociclib, ribociclib and abemaciclib, mTOR inhibitors like everolimus, PI3K inhibitors like alpelisib, PARP inhibitors like olaparib and talazoparib

The term “anti-angiogenic drug”, as used herein, is any compound suitable for use in an anti-angiogenic therapy as defined herein. In one embodiment, an angiogenesis-inhibitor is a VEGF-inhibitor, more specifically a compound inhibiting vascular endothelial growth factor A (VEGF-A). In another embodiment, an angiogenesis inhibitor is an inhibitor of angiopoietin-2 (Ang-2). In a further embodiment, the VEGF- or VEGF-A-inhibitor is a recombinant humanized monoclonal antibody. In another, more specific embodiment said VEGF- or VEGF-A-inhibitor is the molecule with the INN bevacizumab.

Tyrosine kinase inhibitors are mainly small molecule agents that inhibits tyrosine kinases. They are a subclass of targeted therapies. Tyrosine kinases are enzymes responsible for the activation of many proteins by signal transduction cascades. The proteins are activated by adding a phosphate group to the protein (phosphorylation), a step that TKIs inhibit. Numerous TKIs aiming at various tyrosine kinases have been generated by the originators of these compounds and proven to be effective anti-tumor agents and anti-leukemic agents. Based on this work imatinib was developed against chronic myelogenous leukemia (CML) and later gefitinib and erlotinib aiming at the EGF receptor. Dasatinib is a Src tyrosine kinase inhibitor that is effective both as a senolytic and as therapy for CML. Sunitinib, an inhibitor of the receptors for FGF, PDGF and VEGF is also based on early studies on TKIs aiming at VEGF receptors. Regorafenib is a dual targeted VEGFR2-TIE2 tyrosine kinase inhibitor that is approved, inter alia, for metastatic colorectal cancer, advanced gastrointestinal stromal tumours and advanced hepatocellular carcinoma. Adavosertib is a Wee1 kinase inhibitor that is undergoing numerous clinical trials in the treatment of refractory solid tumors.

However, toxicities such as myelosuppression, diarrhea, and supraventricular tachyarrhythmia have arisen while attempting to determine the toxicity and effectiveness of the drug. Lapatinib, FDA approved for treatment in conjunction with chemotherapy or hormone therapy, is also currently undergoing clinical trials in the treatment of HER2-overexpressing breast cancers.

According to embodiments of the invention, the immunotherapeutic approach is preferred. Such approach comprises the administration of at least one selected from the group consisting of
immune checkpoint inhibitor,
a bispecific antibody (e.g. BITE) or TCR comprising an immune effector, like a cytokine or an CD3 binding entity
a tumor vaccine
CAR T cell,
TLR agonist.

According to embodiments of the invention, the immune checkpoint inhibitor is a binder, inhibitor or antagonist of at least one selected from the group consisting of
CTLA-4,
PD-1,
PD-L1,
LAG 3,
TIM3,
OX40,
TIGIT,
ICOS,
41BB (CD137),
VISTA,
and/or GITR.

In one embodiment, the immune checkpoint inhibitor is a binder, inhibitor or antagonist of PD-1 or PD-L1.

According to embodiments of the invention, the binder, inhibitor or antagonist is at least one selected from the group consisting of
Antibody,
Modified antibody format,
Antibody derivative or fragment,
Antibody-based binding protein, and/or
Antibody mimetic.

According to embodiments of the invention, the immune checkpoint inhibitor is at least one selected from the group consisting of
Ipilimumab (anti-CTLA-4),
Nivolumab (anti-PD-1),
Pembrolizumab (anti-PD-1),
Cemiplimab (anti-PD-1),
Spartalizumab (anti-PD-1),
Budigalimab (anti-PD1),
Refitanlimab (anti-PD1),
Dostarlimab (anti-PD1)
Atezolizumab (anti-PD-L1),
Avelumab (anti-PD-L1),
Durvalumab (anti-PD-L1),
INCB86550 (anti-PDL1)
Etigilimab (anti-TIGIT),
BGB-A1217 (anti-TIGIT)
BMS-986207 (anti-TIGIT),
AB154 (anti-TIGIT) ASP8374 (anti-TIGIT),
MK 7684 (anti-TIGIT),
Tiragolumab (anti-TIGIT),
INCAGN1949 (anti-OX40),
INCAGN2390 (anti-TIM3),
INCAGN2385 (anti-LAG3),
MCLA-145 (PDL1×CD137), and/or
MEDI5752 (PD1×CTLA4)

According to embodiments of the invention, the neoplastic disease is a solid tumor, preferably at least one selected from the group consisting of
Bladder Cancer
Breast Cancer
Cervical Cancer
Colon & Rectal Cancer
Endometrial Cancer
Gastric cancer
Renal Cancer.
Head and Neck Cancer
Liver Cancer
Melanoma
Mesothelioma
Neuroendocrine Cancer
Non-Small Cell Lung Cancer
Non-Melanoma Skin Cancer
Ovarian Cancer
Pancreatic Cancer
Prostate Cancer
Sarcoma
Small Cell Lung Cancer
Thyroid Cancer, and/or
Germ cell Cancer.

According to embodiments of the invention, the inhibitor of Tryptophan Hydroxylase is at least one selected from the group consisting of
Fenclonine
LP-533401
LP-533401 hydrochloride
LP-521834
LP-534193
LP-615819
Telotristat ethyl
Telotristat etiprate
Telotristat
6-Fluorotryptophan
p-Ethynylphenylalanine (4-Ethynyl-L-phenylalanine)·
p-Ethynylphenylalanine hydrochloride (4-Ethynyl-L-phenylalanine hydrochloride)
LX-1031
PCPA methyl ester hydrochloride
Rodatristat
Rodatristat ethyl
ACT-678689 and/or
one or more antisense oligonucleotides.

In one embodiment, the Tryptophan Hydroxylase is Tryptophan Hydroxylase 1. It has been discussed that Inhibition of Tryptophan Hydroxylase (Expasy identifier: EC 1.14.16.4, also known as Tryptophan Hydroxylase 1 or Tryptophan 5 monoxygenase) may lead to decrease or total abolishment of serotonin synthesis, and thus may provide effective treatment for disorders that are caused by excessive synthesis or release of serotonin, including chemotherapy-induced nausea and vomiting, gastrointestinal disorders, and pulmonary hypertension (Cianchetta et al 2010). The authors also describe a rationale that can be applied to discover or synthesize new Tryptophan Hydroxylase inhibitors. In such rationale, a phenylalanine moiety in a system of aromatic rings binds to the enzyme in a way that is highly similar to the way in which the substrate tryptophan binds. Quingyun et al (2008) also describe a rationale to synthesize new Tryptophan Hydroxylase inhibitors. For this reason, the skilled artisan is capable, without further inventiveness, to find other suitable inhibitors of Tryptophan Hydroxylase that can be used in the context of the present invention.

Furthermore, antisense oligonucleotides (ASO) can be used as an inhibitor of Tryptophan Hydroxylase. ASOs are capable of altering mRNA expression through a variety of mechanisms, including ribonuclease H mediated decay of the pre-mRNA, direct steric blockage, and exon content modulation through splicing site binding on pre-mRNA.

ASO comprise, essentially, single stranded RNA that is complementary to the messenger RNA (mRNA) encoding for the protein of interest, i.e., Tryptophan Hydroxylase. They hybridizes with said mRNA, and thereby block its translation into the protein.

Cis-acting ASOs are transcribed from the opposite strand of the target gene at the target gene locus. They often show high degree or complete complementarity with the target gene. If the cis-acting ASO regulates gene expression by targeting mRNA, it can only target individual mRNA. Upon interactions with the targeting mRNAs, cis-acting ASOs can either block ribosome binding or recruit RNAase to degrade the targeting mRNAs. Consequently, the function of these cis-acting ASOs is to repress translation of the targeting mRNAs. Besides cis-acting ASOs that target mRNAs, there are cis-acting epigenetic silencers and activators.

In terms of epigenetic modification, cis-acting refers to the nature of these ASOs that regulate epigenetic changes around the loci where they are transcribed. Instead of targeting individual mRNAs, these cis-acting epigenetic regulators can recruit chromatin modifying enzymes which can exert effects on both the transcription loci and the neighboring genes.

Trans-acting ASOs are transcribed from loci that are distal from the targeting genes. In contrast to cis-acting ASOs, they display low degree of complementarity with the target gene but can be longer than cis-acting ASOs. They can also target multiple loci. Because of these properties of trans-acting ASOs, they form less stable complexes with their targeting transcripts and sometimes require aids from RNA chaperone protein such as Hfq to exert their functions. Due to the complexity of the trans-acting ASOs, they are currently considered to be less druggable targets.

ASO-based drugs may contain, in their nucleotide sequence, phosphorothioate substitutions and 2′ sugar modifications to inhibit nuclease degradation enabling vehicle-free delivery to cells. Phosphorothioate ASOs can be delivered to cells without the need of a delivery vehicle. ASOs do not penetrate the blood brain barrier when delivered systemically but they can distribute across the neuraxis if injected in the cerebrospinal fluid typically by intrathecal administration. Newer formulations using conjugated ligands greatly enhances delivery efficiency and cell-type specific targeting.

With Mipomersen, Inotersen, and Givlaari, several ASO-based drugs have been approved for use in recent years. Mipomersen a lipid lowering agent that targets the ApoB gene was eventually approved by the United States Food and Drug Administration (FDA) in 2013. Inotersen (antisense) is targeting transthyretin (TTR) for the treatment of polyneuropathy caused by hereditary transthyretin-mediated amyloidosis, while Givlaari targets aminolevulinic acid synthase 1 (ALAS1) for the treatment of acute hepatic porphyria.

In order to facilitate the sequence design of ASOs, the skilled person as access to a large array of tools. For example, PFRED is a software application for the design, analysis, and visualization of antisense oligonucleotides (Sciabola et al. 2021, the content of which is incorporated herein by reference for enablement purposes). The software provides an intuitive user-interface for scientists to design a library of antisense oligonucleotides that target a specific gene of interest. Moreover, the tool facilitates the incorporation of various design criteria that have been shown to be important for stability and potency. PFRED has been made available as an open-source project so the code can be easily modified to address the future needs of the oligonucleotide research community. A compiled version is available for downloading at https://github.com/pfred/pfred-gui/releases/tag/v1.0 as a java Jar file.

The tool provides a user-friendly interface where the only required input is an accession number for the target gene, the tool returns a list of properties that contribute to the efficacy of an ASO. These properties include human transcripts and cross-species homology, GC content, SNPs, intron-exon boundary, duplex thermodynamics, efficacy prediction score and off-target matches. An automated oligonucleotide selection procedure is available to quickly select one potential set of sequences with an appropriate property profile. The selection protocol can be customized by the user through changes of the selection cutoffs or the addition of alternate design parameters and algorithms.

Full guidance for experiments using antisense oligonucleotides is provided in Gagnon and Corey (2019), the content of which is incorporated herein by reference for enablement purposes.

As discussed above, the accession numbers of the respective enzymes are shown in the below table. Hence, the skilled person is fully capable of designing ASOs on the basis of the disclosure provided herein and common general knowledge.

TABLE 3
Accession numbers of the genes encoding
for the enzymes of interest

| Enzyme | Gene ID |
|---|---|
| Tryptophan Hydroxylase 1 | 7166 (Homo sapiens) |
| Tryptophan Hydroxylase 2 | 121278 (Homo sapiens) |
| 5-HTP decarboxylase | |
| Arginase 1 | 383 (Homo sapiens) |
| Arginase 2 | 384 (Homo sapiens) |
| | 100129854 (Homo sapiens) |
| Nitric oxide synthase 1 (NOS 1) | 4842 (Homo sapiens) |
| Nitric oxide synthase 2 (NOS 2) | 4843 (Homo sapiens) |
| Nitric oxide synthase 3 (NOS 3) | 4846 (Homo sapiens) |

According to embodiments of the invention, the inhibitor of 5-HTP decarboxylase is at least one selected from the group consisting of
Ro 4602, and/or
one or more antisense oligonucleotides.

Inhibitors of 5-HTP decarboxylase (Expasy identifier: EC 4.1.1.28, alternative names: aromatic-L-amino-acid decarboxylase, 5-hydroxytryptophan decarboxylase, aromatic amino acid decarboxylase, DOPA decarboxylase, Hydroxytryptophan decarboxylase, L-DOPA decarboxylase, Tryptophan decarboxylase) are well known in the art

For this reason, the skilled artisan is capable, without further inventiveness, to find other suitable inhibitors of Tryptophan e Hydroxylase that can be used in the context of the present invention.

Furthermore, antisense oligonucleotides (ASO) can be used as an inhibitor of 5-HTP decarboxylase. See the above discussion for further technical background and enablement.

According to embodiments of the invention, the inhibitor of Arginase is at least one selected from the group consisting of
Resveratrol
Norvaline
N-hydroxy-l-arginine
N-hydroxy-nor-L-arginine (nor-NOHA)
N-hydroxy-guanidinium
2 (S)-amino-6-boronohexanoic acid (ABH)
S-(2-boronoethyl)-L-cysteine (S-2-BEC),
(R)-2-amino-6-borono-2-(2-(piperidin-1-yl)ethyl) hexanoic acid, and/or
one or more antisense oligonucleotides.

Inhibitors of arginase (Expasy identifier: EC 3.5.3.1) have been in the scientific discussion for decades, in particular when in 1990s nitric oxide (NO) was reported as one of the most important biological mediators and its role in cardiovascular diseases was broadly discussed (Loscalzo and Welch, 1992). As a result, the development of arginase inhibitors was accelerated.

The finding of arginase inhibitors has been subject to rational design, as e.g. disclosed in Steppan et al (2013) or Pudlo et et al (2017). For this reason, the skilled artisan is capable, without further inventiveness, to find other suitable arginase inhibitors that can be used in the context of the present invention.

Furthermore, antisense oligonucleotides (ASO) can be used as an inhibitor of arginase. See the above discussion for further technical background and enablement.

According to embodiments of the invention, the inhibitor of Nitric oxide synthase is at least one selected from the group consisting of:
L-NANA (L-NG-Methyl-L-arginine)
N-PLA (L-NG-Propyl-L-arginine)
L-NNA (L-NG-Nitroarginine)
L-NAME (L-NG-Nitroarginine methyl ester)
L-NAA NG-Amino-L-arginine
ADMA (NG,NG-Dimethyl-L-arginine)
SDMA (NG,NG′-Dimethyl-L-arginine)
L-NIL (L-N6-(1-Imino-ethyl) lysine)
L-Thiocitrulline
S-Methyl-L-Thiocitrulline
Agmatine (1-Amino-4-guanidinobutane)
L-Canavanine
L-NMMA (Tilarginine)
N(G)-methyl-l-arginine hydrochloride (546C88)
Ng-nitro-l-arginine (L-NArg)
AMIDINES
L-NIO Nδ-(Iminoethyl)-L-ornithine
Ethyl-L-NIO
Vinyl-L-NIO
1400W (N-(3-(Aminomethyl)benzyl) acetamidine)
INDAZOLE DERIVATES
7-NI (7-Nitroindazole)
7-NI-Br (7-Bromonitroindazole)
Imidazole derivates
TRIM (1-[2-(Trifluoromethyl)phenyl-imidazole
2-IMINOPIPERIDINE DERIVATES
2-Imino-4-methylpiperidine
HYDRAZINE DERIVATES
Aminoguanidine
ISOTHIOUREAS
S-(2-Aminoethyl) isothiourea
1,3-PBIT (S,S′-(1,3-Phenylene-bis(1,2-ethanediyl))bis-isothiourea)
1,4-PBIT (S,S′-(1,4-Phenylene-bis(1,2-ethanediyl))bis-isothiourea)
α-Guanidinoglutaric Acid,
Methylene blue,
ODQ ([1H-[1,2,4]Oxadiazole [4,3-a]quinoxalin-1-one], and/or
one or more antisense oligonucleotides.

Inhibitors of nitric oxide synthase (Expasy identifiers: EC 1.14.13.39, 1.14.14.47) have also been disclosed in the prior art. Also in the 1990s, excessive nitric oxide production was recognized in septic shock and cardiogenic shock. Nitric oxide synthase inhibitors were found to revert hypotension in shock induced by cytokines, heat and hemorrhage in animal studies. Nitric oxide synthase inhibitors were therefore thought to be promising agents for these conditions (Wong & Lerner 2015). There are three isoforms of Nitric oxide sybthase (NOS). eNOS (endothelial NOS) and nNOS (neuronal NOS) are constitutively expressed and regulated by transcription and post-transcription processes. iNOS (inducible NOS) is released de novo in response to inflammation. NOS inhibitors of varying degrees of potency and selectivity are available and utilized in research studies. NOS inhibitors of varying degrees of potency and selectivity are available and utilized in research studies (Wong & Lerner 2015). They have also been discussed to serve as antidepressants (Wegener & Volke 2010). For these reason, the skilled artisan is capable, without further inventiveness, to find other suitable Nitric oxide synthase inhibitors that can be used in the context of the present invention.

Furthermore, antisense oligonucleotides (ASO) can be used as an inhibitor of nitric oxide synthase. See the above discussion for further technical background and enablement.

According one embodiment of the invention, the inhibitor of Tryptophan Hydroxylase is Telotristat ethyl, Telotristat etiprate, or Telotristat.

The inhibition of Tryptophan Hydroxylase by Telotristat may lead to decrease or total abolishment of serotonin (5HT) synthesis. See the above discussion for further background and enablement.

Telotristat ethyl is a prodrug of telotristat. As used herein, the term “prodrug” refers to a pharmacologically inactive compound that, after intake, is metabolized (i.e., converted within the body) into a pharmacologically active drug. Instead of administering a drug directly, a corresponding prodrug can be used to improve how the drug is absorbed, distributed, metabolized, and/or excreted. After absorption, the prodrug is rapidly hydrolysed by carboxylesterases to the active substance telotristat. Telotristat ethyl may be formulated as telotristat etiprate, which is a hippurate salt of telotristat ethyl.

According one embodiment of the invention, the inhibitor of Nitric oxide synthase is L-NMMA (Tilarginine).

Tilarginine, also called L-N-monomethyl arginine (L-NMMA), is a non-specific inhibitor of nitric oxide synthase (NOS). Upon administration, tilarginine binds to and inhibits NOS leading to a reduction in NOS activity that may abrogate the immunosuppressive TME, enhance tumor antigen-specific immune response and/or inhibit tumor cell proliferation.

According to another aspect of the invention, a kit for carrying out a method according to the above description is provided, said kit comprising means for detecting at least one metabolite selected from the group consisting of Arginine, Tryptophan, 5-HT, 5-HTP, Citrulline and/or Ornithine in an immunoassay.

In one embodiment, such immunoassay is a quantitative immunoassay. Quantitative immunoassays are used to measure the level or concentration of an analyte, like here a metabolite, in a biologic sample, such as plasma or serum. Immunoassays are based on the specific binding ability of binder, like e.g. an antibody, to such analyte.

By comparing the percentage of bound analyte (% [bound/total]) from a sample to the dose-response curve generated by known concentrations of the analyte, the amount of analyte in the biologic sample can be quantified.

According to one embodiment, said means for detecting at least one metabolite comprises at least one binder specifically binding to such metabolite.

In embodiments, such binder is a protein binder or another binder selected from the group consisting of
a polyclonal or monoclonal antibody, or fragment or derivative thereof,
a new antibody format
a fusion peptide, and/or
an antibody mimetic.

the term “monoclonal antibody (mAb)” shall refer to an antibody composition having a homogenous antibody population, i.e., a homogeneous population consisting of a whole immunoglobulin, or a fragment or derivative thereof. Particularly preferred, such antibody is selected from the group consisting of IgG, IgD, IgE, IgA and/or IgM, or a fragment or derivative thereof.

As used herein, the term “fragment” shall refer to fragments of such antibody retaining, in some cases, target binding capacities, e.g.
a CDR (complementarity determining region)
a hypervariable region,
a variable domain (Fv)
an IgG heavy chain (consisting of VH, CH1, hinge, CH2 and CH3 regions)
an IgG light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs. All these items are explained below.

Methods for the production and/or selection of chimeric, humanised and/or human mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in US200302048621 by TaconicArtemis.

IgG, scFv, Fab and/or F (ab) 2 are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine(S) or glycine (G). This chimeric molecule retains the specificity of the original immunoglobulinThe term “new antibody formats” encompasses, for example bi- or trispecific antibody constructs, Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g., antibody or fragments or derivatives linked to a toxin, a cytokine, a radioisotope or a label). This list is however not restrictive.

Antibodies which bind to the metabolites as discussed above are for example available at Immusmol SAS, Bordeaux, under the following catalogue numbers:

TABLE 4a
examples of diagnostic antibodies to be
used in the context of the invention

| | Type | |
|---|---|---|
| | mAb = monoclonal, | Catalogue |
| antibody | pAb = polyclonal | number |
| Anti-Tryptophan | mouse mAb | #IS011 |
| Anti-5 Hydroxytryptophan | rabbit pAb | #IS1081-2 |
| Anti-5-Hydroxytryptamine | mouse mAb | #IS040 |
| | rabbit pAb | #IS1007 |
| Anti-Arginine | rabbit pAb | #IS1055 |
| Anti-Ornithine | rabbit pAb | #IS1059 |
| Anti-Citrulline | rabbit pAb | #IS1067 |

Antibodies which bind to the enzymes as discussed above are for example available under the following catalogue numbers (just a random selection, many other suitable antibodies are available by many different suppliers)

TABLE 4b
examples of diagnostic antibodies to be
used in the context of the invention

| | Type | | |
|---|---|---|---|
| | mAb = monoclonal, | | Catalogue |
| antibody | pAb = polyclonal | supplier | number |
| Anti-TPH1 | rabbit pAb | Thermo | PA1-777 |
| | | Fisher | |
| Anti-TPH2 | Goat pAb | abcam | ab121013 |
| 5-HTP | | | |
| decarboxylase | | | |
| Anti-arginase 1 | tat mAb | Invitrogen | A1exF5 |
| Anti-arginase 2 | rabbit pAb | Cell Signaling | #19324 |
| | | technology | |
| Anti-NOS | rabbit pAb | Antikörper- | ABIN1838225 |
| | | online.de | |

The term “fusion peptide” or “fusion protein” proteins relates, for example, to proteins consisting of an immunoglobulin Fc portion plus a target binding moiety capable of binding an enzyme and/or a metabolite of the kynurenine pathway (so-called-cept molecules).

The term “antibody mimetic” relates to target binding proteins, which are not related to immunoglobulins. Many of the above-mentioned techniques, like phage display, are applicable for these molecules as well. Such antibody mimetics are for example derived from Ankyrin Repeat Proteins, C-Type Lectins, A-domain proteins of Staphylococcus aureus, Transferrins, Lipocalins, Fibronectins, Kunitz domain protease inhibitors, Ubiquitin, Cysteine knots or knottins, thioredoxin A, and so forth, and are known to the skilled person in the art from the respective literature.

In embodiments, the binder is labelled. Such label may consist, e.g., of a radioisotope, an enzyme, a luminescent entity, a fluorescent entity, a phosphorescent entity, a metal-containing particle (e.g., a gold-containing particle), an X-ray dense entity or the like.

According to one embodiment, said immunoassay is at least one selected from the group consisting of
Immunochemistry (IC),
Enzyme Immunoassays (EIA) or Enzyme-linked immunosorbent assays (ELISA),
Immunocytochemistry assay (ICC), and/or
Immunofluorescence (IF).

According to another aspect of the invention, a drug combination (in the manufacture of one or more medicaments) for use in the treatment of a patient being diagnosed for, suffering from or being at risk of developing cancer is provided, said patient being characterized as having, as determined in one or more samples collected from said patient, a high activity of at least one of Tryptophan Hydroxylase, 5-HTP decarboxylase, Arginase and/or Nitric oxide synthase, which drug combination comprises
(i) anti-cancer therapy, and
(ii) an inhibitor of Tryptophan Hydroxylase, 5-HTP decarboxylase, Arginase and/or Nitric oxide synthase

This language is deemed to encompass both the swiss type claim language accepted in some countries (in this case, brackets are deemed absent) and EPC2000 language (in this case, brackets and content within the brackets is deemed absent).

As regards this aspect, the preferred embodiments discussed elsewhere herein regarding
the surrogate measurements (ratio [5-HT/Trp], [5-HTP/Trp], [5-HT/5-HTP], [Orn/Arg] and [Cit/Arg])
the threshold levels
the tumor types, and
the types of therapy
   apply mutatis mutandis.

According to another aspect of the invention, A method of treating a patient being diagnosed suffering from or being at risk of developing
cancer, said patient being characterized as having, as determined in one or more samples collected from said patient, a low activity of at least one of Tryptophan Hydroxylase, 5-HTP decarboxylase, Arginase and/or Nitric oxide synthase, wherein the method comprises, administration of
   (i) anti-cancer therapy, and
   (ii) an inhibitor of Tryptophan Hydroxylase, 5-HTP decarboxylase, Arginase and/or Nitric oxide synthase
in one or more therapeutically effective doses.

As regards this aspect, the preferred embodiments discussed elsewhere herein regarding
the surrogate measurements (ratio [5-HT/Trp], [5-HTP/Trp], [5-HT/5-HTP], [Orn/Arg] and [Cit/Arg])
the threshold levels
the tumor types, and
the types of therapy
   apply mutatis mutandis.

In any case, the drug combination can be administered to the patient in a single combined dosage unit, or in separate dosage units. Administration can take place simultaneously or one after the other, in any conceivable order, with pauses, interruptions, intervals or breaks in between if applicable.

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Example 1

The MCA205 Mouse Fibrosarcoma Cell Line was cultured in vitro according to Merck Millipore specifications. To derive a clone resistant to anti-PD1 immunotherapy, a cell suspension was prepared according to the viable cell count and was inoculated into the flank of C57BL/6J mice purchased from Charles River (L'Abresle, France). Anti-PD-1 monoclonal antibody (clone RMP1-14—BioXCell) was administered intraperitoneally at 5 mg/kg and treatments were repeated 4 times, on days 6, 9, 12, and 15.

Tumors from non-responsive mice were collected, dissociated and cultured in vitro. A mouse is defined as non-responder if its death occurs in the same time as all the vehicle-treated mice. The obtained resistant MCA205 cells (MCA205-PD1R) were inoculated again into C57BL/6J mice and treated with anti-PD-1, for 2 successive allograft experiments.

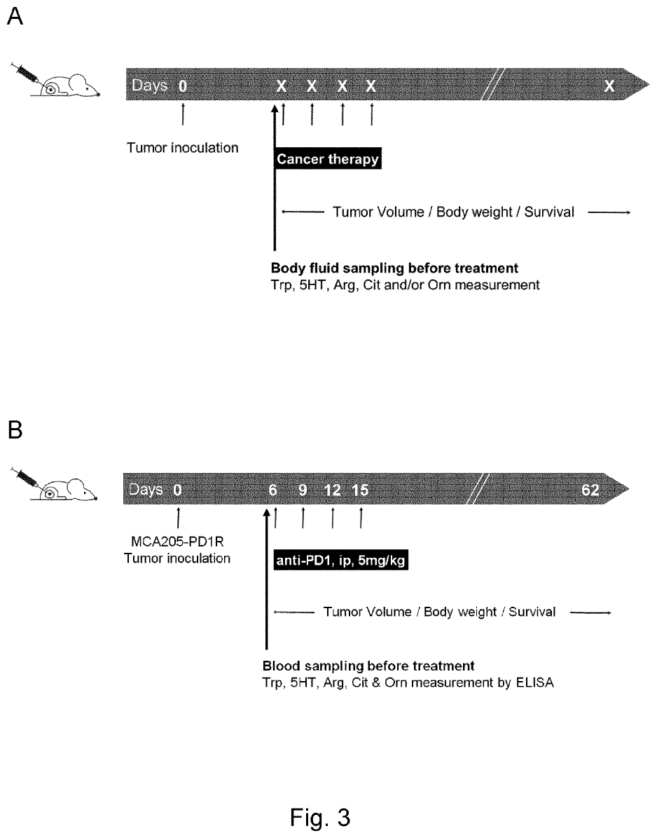

After the third round of resistant clone selection, a MCA205 resistant cell line (“MCA205-PDIR”) was defined and inoculated into C57BL6/J mice (day 0 in FIG. 3). Mice were further treated with anti-PD1 antibody (clone RMP1-14-BioXCell) on Days 6, 9, 12 and 15. Starting from day 6 post tumor cell inoculation, all experimental animal groups were monitored 3 times a week for body weight, survival and tumor volume measured by physical examination and according to the formula “V=Length*Width2/2”.

One day prior treatment initiation, blood samples were collected through retro-orbital puncture. The plasma was processed and stored at −80° C. until amino acid/metabolite quantification by ELISA. Mice were finally classified as “High” or “Low” for each amino acid/metabolite ratio/quotient based on an optimized threshold obtained using the maximally selected rank statistics from the maxstat R package and using the overall survival as optimal outcome (survminer R package v0.4.9).

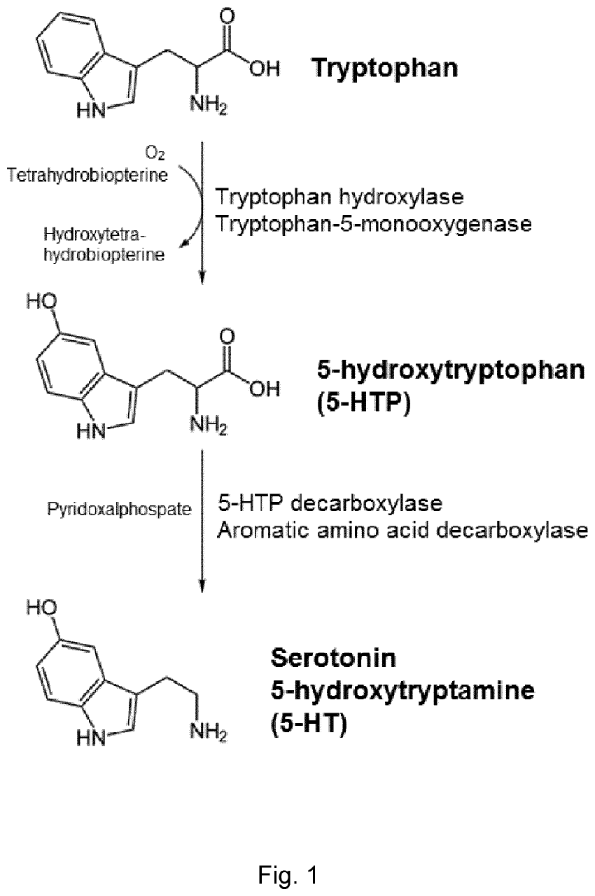

FIG. 1 shows the tryptophan degradation pathway.

A high ratio of the levels (concentration, titer) of 5-HT and Trp (5-HT/Trp) indicates a low level (concentration, titer) of Trp, and thus a high activity of the enzymes that convert Trp to 5-HTP and 5-HT, i.e., Tryptophan Hydroxylase and/or 5-HTP decarboxylase.

A high ratio of the levels (concentration, titer) of 5-HTP and Trp (5-HTP/Trp) indicates a low level (concentration, titer) of Trp, and thus a high activity of the enzyme that converts Trp to 5-HTP, i.e., Tryptophan Hydroxylase.

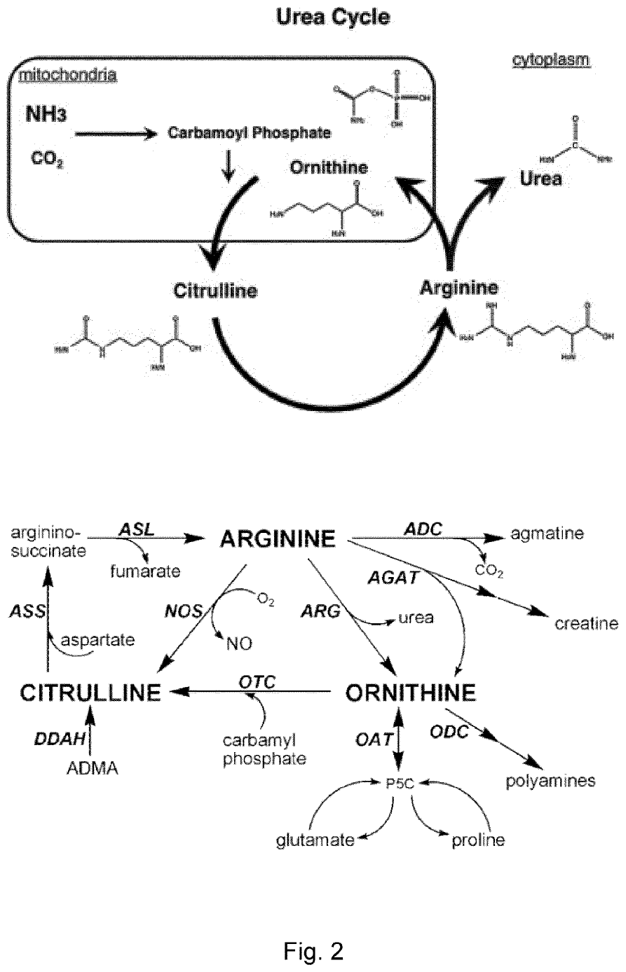

FIG. 2 shows the urea cycle.

A high ratio of the levels (concentration, titer) of Ornithine (Orn) and Arginine (Arg) (Orn/Arg) indicates a low level (concentration, titer) of Arg, and thus a high activity of the enzyme that converts Arg to Orn, i.e., Arginase.

A high ratio of the levels (concentration, titer) of Citrulline (Cit) and Arginine (Arg) (Cit/Arg) indicates a low level (concentration, titer) of Arg, and thus a high activity of the enzyme that converts Arg to Cit, i.e., Nitric Oxide Synthase.

FIG. 3 shows the experimental protocol that is underlying example 1. MCA205-PDIR cells derived from a mouse sarcoma model that is resistant to anti-PD1 and anti-PDL1 were inoculated into C57BL6/J mice. One day prior treatment initiation, blood samples were collected through retro-orbital puncture. The plasma was processed and stored at −80° C. until amino acid/metabolite quantification by ELISA. Mice were further treated with anti-PD1 antibody (clone RMP1-14-BioXCell) on Days 6, 9, 12 and 15. Starting from day 6 post tumor cell inoculation, all experimental animal groups were monitored 3 times a week for body weight, survival and tumor volume measured by physical examination and according to the formula “V=Length*Width2/2”. Mice were finally classified as “High” or “Low” for each amino acid/metabolite ratio/quotient based on an optimized threshold obtained using the maximally selected rank statistics from the maxstat R package and using the overall survival as optimal outcome (survminer R package v0.4.9).

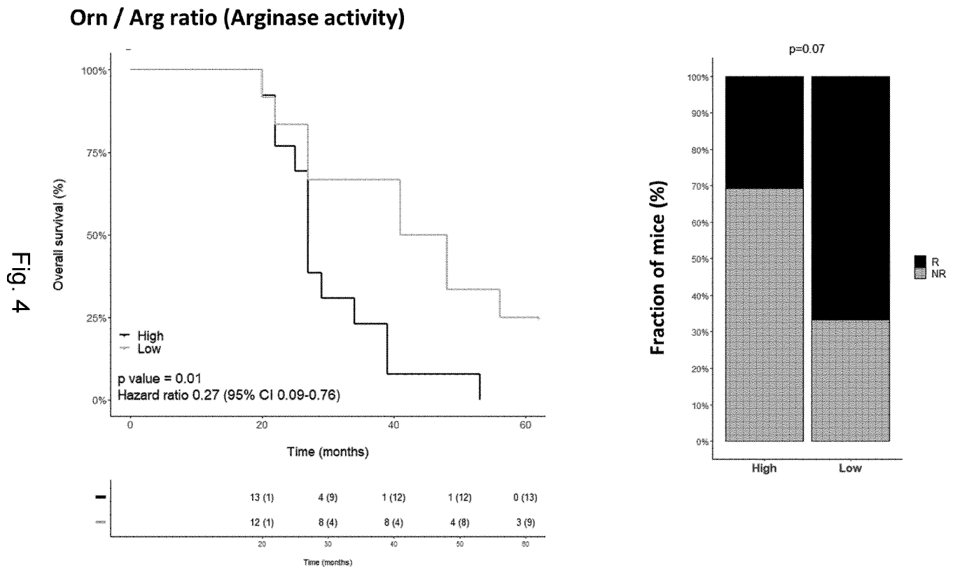

FIG. 4 shows Kaplan Meier curves for mice treated according to example 1, in which the ratio of Ornithine to Arginine was determined, as a surrogate for the Arginase activity per se. A high ratio was considered to represent a high Arginase activity and a low Arginine level, whereas a low ratio was considered to represent a low Arginase activity and a high Arginine level. The thresholds are as shown in table 2.

It can be seen that mice with high Orn/Arg ratio (=low Arginine level/high Arginase activity) have a worse survival, when treated with a PD-1 inhibitor, than mice with a low ratio (=high Arginine level/low Arginase activity). Hence, mice with a high ratio (=low Arginine level/high Arginase activity) could benefit from increased Arginine levels, as for example caused by administration of an inhibitor of Arginase.

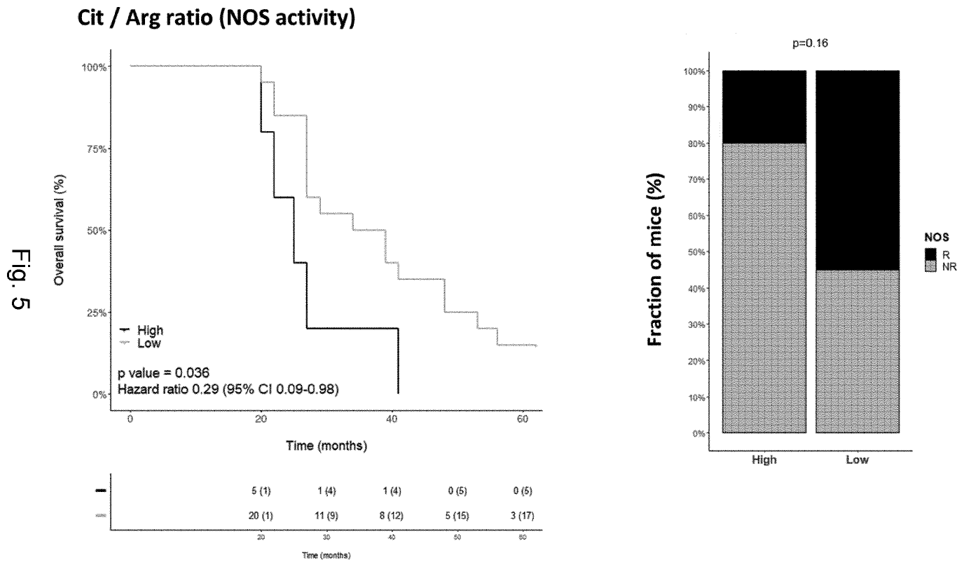

FIG. 5 shows Kaplan Meier curves for mice treated according to example 1, in which the ratio of Citrulline and Arginine was determined, as a surrogate for the Nitric Oxyde Synthase activity. A high ratio was considered to represent a low Arginine level/high Nitric Oxide Synthase activity, whereas a low ratio was considered to represent a high Arginine level/low Nitric Oxide Synthase activity. The thresholds are as shown in table 2.

It can be seen that mice with high Cit/Arg ratio (=low Arginine level/high Nitric Oxide Synthase activity) have a worse survival, when treated with a PD-1 inhibitor, than mice with a low ratio (=high Arginine level/low Nitric Oxide Synthase activity). Hence, mice with a high ratio (=low Arginine level/high Nitric Oxide Synthase activity) could benefit from increased Arginine levels, as for example caused by administration of an inhibitor of Nitric oxide synthase.

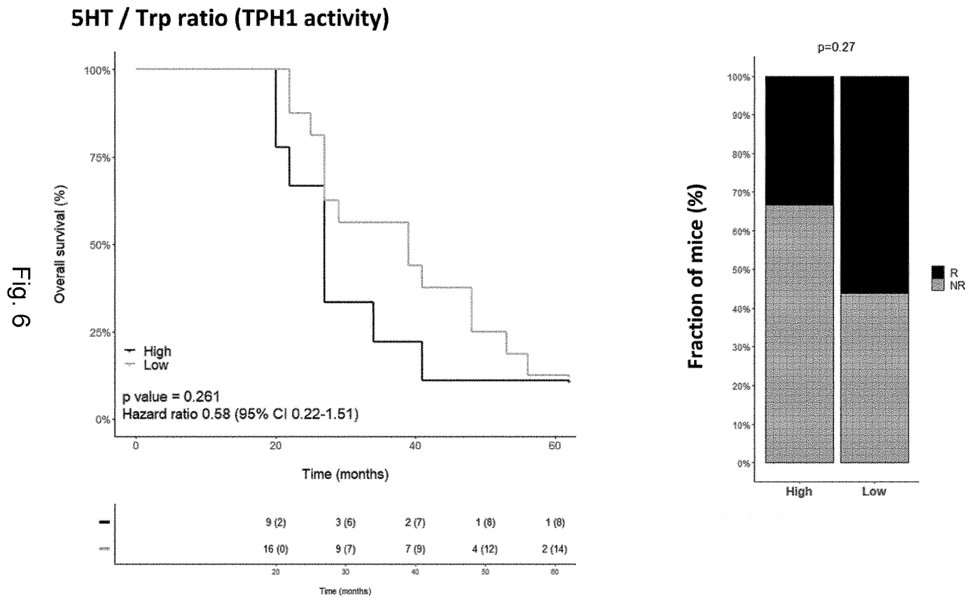

FIG. 6 shows Kaplan Meier curves for mice treated according to example 1, in which the ratio of 5-HT to Tryptophan was determined, as a surrogate for the Tryptophan level per se and/or the Tryptophan hydroxylase activity and/or 5-HTP decarboxylase activity. A high ratio was considered to represent a low Tryptophan level (=high Tryptophan hydroxylase activity and/or 5-HTP decarboxylase activity), whereas a low ratio was considered to represent a high Tryptophan level (=low Tryptophan hydroxylase activity and/or 5-HTP decarboxylase activity). The thresholds are as shown in table 2.

It can be seen that mice with high 5HT/Trp ratio (=low Tryptophan level/high Tryptophan hydroxylase activity and/or 5-HTP decarboxylase activity,) have a worse survival, when treated with a PD-1 inhibitor, than mice with a low ratio (=high Tryptophan level/low Tryptophan hydroxylase activity and/or 5-HTP decarboxylase activity). Hence, mice with a high ratio (=low Tryptophan level/high Tryptophan hydroxylase activity and/or 5-HTP decarboxylase activity) could benefit from increased Tryptophan levels, as for example caused by administration of an inhibitor of Tryptophan hydroxylase or an inhibitor of 5-HTP decarboxylase.

### Example 2

### Quantification of Plasmatic Tryptophan Metabolites by HPLC

Plasma concentrations of L-Tryptophan (Trp), 5-hydroxTryptophan (5HTP) and Serotonin (5HT) were measured with a targeted metabolomic approach from plasma samples collected from healthy subjects and cancer patients. Levels of metabolites were measured by high-performance liquid chromatography (HPLC) (Waters® (Millford, MA, USA)). Calibration curves and standards were assessed by using 100 μL of serum samples and 100 μL of a solution of internal standards and 300 μL of methanol. After stirring and incubation for 30 min at −20° C., each sample was centrifuged (15,000 g for 15 min at 4° C.) and the resulting supernatant (300 μL) was transferred to 96-well plates. After simultaneous evaporation, each well was resuspended in 100 μL of a methanol/water mixture (10/90). Finally, 5 μL were injected into the HPLC including a Kinetex C18 xb column (1.7 μm×150 mm×2.1 mm, temperature 55° C.) associated with a gradient of two mobile phases (Phase A: Water+0.5% formic acid; Phase B: MeOH+0.5% formic acid) at a flow rate of 0.4 mL/min. The 5HTP/Trp and 5HT/Trp ratios, which serves as a surrogate of TPH1 activity, were finally calculated.

### Quantification of Plasmatic Arginine Metabolites by UPLC-MS/MS

Plasma concentrations of L-Arginine (Arg), Ornithine (Orn) and Citrulline (Cit) were measured with a targeted metabolomic approach from plasma samples collected from healthy subjects and cancer patients. Levels of metabolites were measured by Ultra-performance liquid chromatography (UPLC) coupled with tandem mass spectrometry (MS/MS) (Waters® (Millford, MA, USA)). Calibration standards and samples were independently assessed by mixing 5 μL of the solution with 45 μL of Acetonitrile. The solutions were finally vortexed prior analysis by UPLC-MS/MS which includes a ACQUITY UPLC BEH Amide Column (1.7 μm×150 mm×1 mm, temperature 50° C.) associated with a gradient of two mobile phases (Phase A: Water+0.1% formic acid; Phase B: Acetonitrile+0.1% formic acid) at a flow rate of 0.6 mL/min. The mass spectrometer was set to a positive electrospray ionization mode with multiple reaction monitoring (MRM). The Cit/Arg and Orn/Arg ratios, which serves as a surrogate of Nitric oxide Synthase and Arginase activity, respectively, were finally calculated.

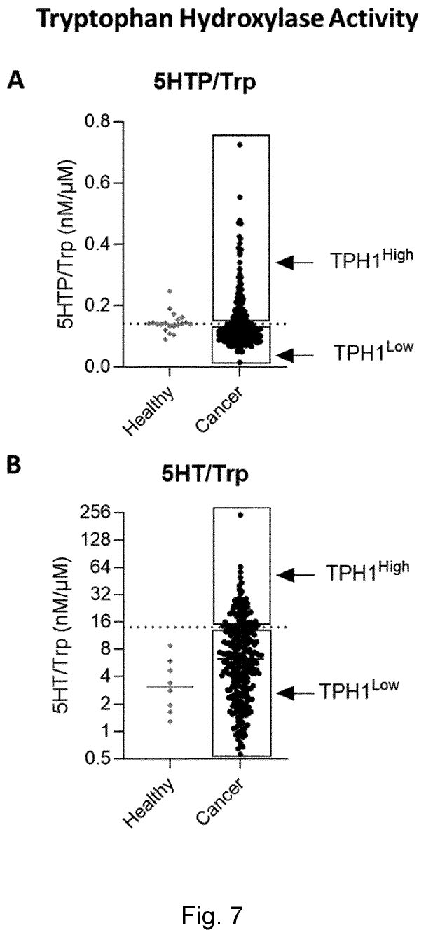

FIG. 7 shows the evaluation of Tryptophan Hydroxylase (TPH1) activity evaluated in plasma from human healthy subjects (FIG. 7A: n=20; FIG. 7B: n=8) and patients suffering from different type of cancer including Melanoma (n=24), Cutaneous Squamous Cell Carcinoma (n=11), Triple Negative Breast cancer (n=9), Urothelial Cancer (n=15), Clear Cell Renal Carcinoma (n=15), Head and neck Squamous Cell Carcinoma (n=11), Sarcoma (n=22), Small cell Lung cancer (n=6), Non-Small Cell Lung Carcinoma (n=106), Ovarian carcinoma (n=5), Gastric Cancer (n=11) and Colorectal cancer (n=11) through mass spectrometry-based quantification of 5-hyfroxyTryptophan (5HTP) to Tryptophan (Trp) ratio (A) or 5-hydroxyTryptamine (5HT) to Trp ratio (B).

As depicted in FIG. 7A, two cancer patient populations can be defined according to the TPH1 activity and the 5HTP (nM) to Trp (μM) ratio: One patient population being characterized by a high TPH1 activity, i.e. above the indicated threshold (TPH1^{High}, threshold=0.14) and one other patient population being characterized by a low TPH1 activity, i.e. below the indicated threshold (TPH1^{Low}, threshold=0.14).

As depicted in FIG. 7B, two cancer patient populations can be defined according to the TPH1 activity and the 5HT (nM) to Trp (μM) ratio: One patient population being characterized by a high TPH1 activity, i.e. above the indicated threshold (TPH1^{High}, threshold=14) and one other patient population being characterized by a low TPH1 activity, i.e. below the indicated threshold (TPH1 Low, threshold=14).

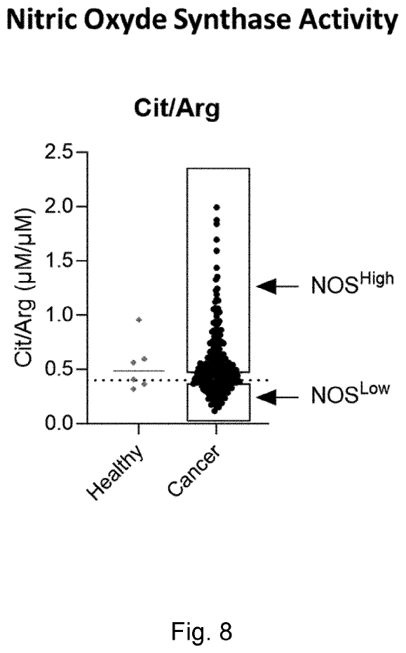

FIG. 8 shows the evaluation of Nitric Oxyde Syntase (NOS) activity evaluated in plasma from human healthy subjects (n=6) and patients suffering from different type of cancer including Melanoma (n=24), Cutaneous Squamous Cell Carcinoma (n=11), Triple Negative Breast cancer (n=9), Urothelial Cancer (n=15), Clear Cell Renal Carcinoma (n=15), Head and neck Squamous Cell Carcinoma (n=11), Sarcoma (n=22), Small cell Lung cancer (n=6), Non-Small Cell Lung Carcinoma (n=106), Ovarian carcinoma (n=5), Gastric Cancer (n=11) and Colorectal cancer (n=11) through mass spectrometry-based quantification of Citruline (Cit) to Arginine (Arg) ratio.

As depicted in FIG. 8, two cancer patient populations can be defined according to the NOS activity, and the Citruline (μM) to Arginine (μM) ratio. One patient population being characterized by a high NOS activity, i.e. above the indicated threshold (NOS^{High}, threshold=0.4) and one other patient population being characterized by a low NOS activity, i.e. below the indicated threshold (NOS^{Low}, threshold=0.4).

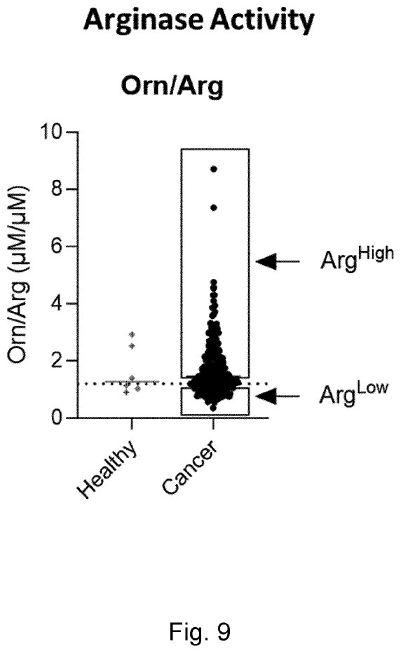

FIG. 9 shows the evaluation of Arginase activity evaluated in plasma from human healthy subjects (n=6) and patients suffering from different type of cancer including Melanoma (n=24), Cutaneous Squamous Cell Carcinoma (n=11), Triple Negative Breast cancer (n=9), Urothelial Cancer (n=15), Clear Cell Renal Carcinoma (n=15), Head and neck Squamous Cell Carcinoma (n=11), Sarcoma (n=22), Small cell Lung cancer (n=6), Non-Small Cell Lung Carcinoma (n=106), Ovarian carcinoma (n=5), Gastric Cancer (n=11) and Colorectal cancer (n=11) through mass spectrometry-based quantification of Ornithine (Orn) to Arginine (Arg) ratio.

As depicted in FIG. 9, two cancer patient populations can be defined according to the Arginase activity and the Ornithine (μM) to Arginine (μM) ratio: One patient population being characterized by a high Arginase activity, i.e. above the indicated threshold (Arginase^{High}, threshold=1.2) and one other patient population being characterized by a low Arginase activity, i.e. below the indicated threshold (Arginase^{Low}, threshold=1.2).

### Example 3

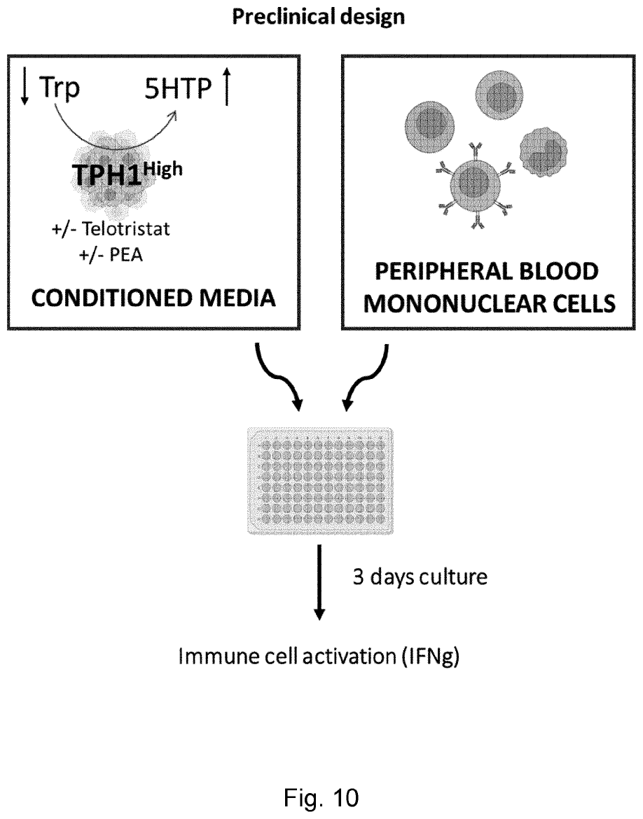

The impact of TPH1^{High} conditioned culture media (C-Media) on immune cell activation was evaluated. The experimental design is shown in FIG. 10. QGP1 neuroendocrine tumor cell line characterized by a high TPH1 expression and activity was cultured over a 48-hour period in the presence and absence of Telotristat (TPH1 inhibitor) or Phenethylamine (PEA) alone and in the presence of Telotristat.

The media characteristics were as follows:
i) NC-Media
ii) C-Media
iii) C-Media+Telotristat (TPH1 inhibitor)
iv) C-Media+Phenethylamine (PEA)
v) C-Media+Telotristat (TPH1 inhibitor)+Phenethylamine (PEA)

Culture supernatant was then collected and applied on peripheral blood mononuclear cells (PBMCs) cultivated under anti-CD3-based activation for 3-days.

### Cell Culture and Effect of Conditioned Media

The QGP-1 cell line was cultured in RPMI-1640 medium, supplemented with 10% (v/v) FCS and penicillin-streptomycin in a humidified incubator at 5% CO₂ and 37° C. In order to prepare the conditioned media, cells were cultured in 6-well plates for 48 hours in the presence and absence of Telotristat (1 μM), a TPH1 inhibitor, and in the presence and absence of Phenethylamine (PEA; 300 μM), a TPH1 inducer (Zhai et al., 2023). QGP1-conditioned media (C-Media) were used to culture human PBMCs (Peripheral Blood Mononuclear Cells) originating from one healthy donor for a 72-hour period under anti-CD3 activation (0.5 μg/mL). As control, fresh cell culture media (Non-Conditioned Media (NC-Media) was used.

### Quantification of Tryptophan Metabolites by Immuno-Assay in Cell Culture Supernatants

Each analytes-Tryptophan (Trp), 5-hydroxyTryptopan (5HTP) and Serotonin (5HT)-were measured individually and then used to calculate ratio, i.e. 5HTP/Trp and 5HT/Trp as representative surrogate of TPH1 activity.

### Trp Quantification

Tryptophan was quantified using an immunoassay (#BA-E-2700, ImmuSmol, Bordeaux, FRANCE). Briefly, in order to remove proteins, 20 μL of sample were precipitated, vortexed and then centrifuged for 15 minutes (3,000 g, at 4° C.). Twenty five microliters of supernatant were then used for derivatization for a 2 hour period at room temperature. Once the derivatization was achieved, 25 μL of the solution was applied on a 96-microtiters prior addition of 50 μL of anti-Tryptophan antiserum. Plate was then incubated overnight at 4° C. After washes and addition of HRP-conjugated secondary antibody for 30 minutes at room temperature, substrate was added for 20-30 minutes. As a competitive ELISA, optical density was inversely correlated with the Tryptophan concentration.

### 5HTP Quantification

5-HdroxyTryptophan was quantified using an immunoassay. Briefly, 100 μL of sample were applied on an extraction plate under acidic condition and washed three times before sample derivatization for a 2 hour period at room temperature. Once the derivatization was achieved, 50 μL of the solution was applied on a 96-microtiters prior addition of 50 μL of anti-5HTP antiserum. Plate was then incubated overnight at 4° C. After washes and addition of HRP-conjugated secondary antibody for 30 minutes at room temperature, substrate was added for 20-30 minutes. As a competitive ELISA, optical density was inversely correlated with the Tryptophan concentration.

### 5HT Quantification

Serotonin was quantified using an immunoassay (#BA-E-5900R, ImmuSmol, Bordeaux, FRANCE). Briefly, 25 μL of sample were mixed with 75 μL diluent and subjected to derivatization through the use of 25 μL acylation reagent for 30 minutes. Once the derivatization was achieved, 100 μL of the solution was applied on a 96-microtiters prior addition of 25 μL of anti-Serotonin antiserum. Plate was then incubated overnight at 4° C. After washes and addition of HRP-conjugated secondary antibody for 30 minutes at room temperature, substrate was added for 20-30 minutes. As a competitive ELISA, optical density was inversely correlated with the Serotonin concentration.

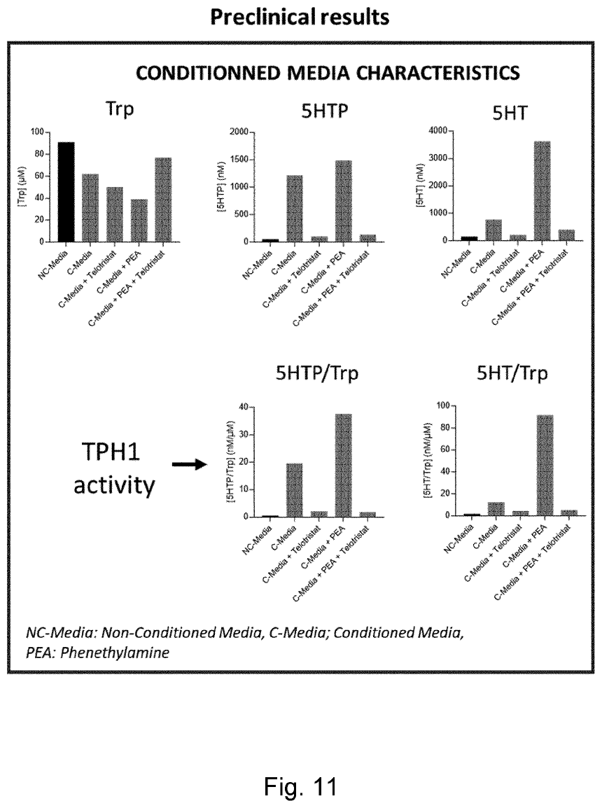

FIG. 11 shows the metabolic characteristics of QGP-1 conditioned media. As depicted in FIG. 11, and when compared to the non-conditioned media (NC-Media), QGP1 conditioned cell culture supernatant (C-Media) with a decrease in Trp level and a concomitant increase of the levels of 5HTP and 5HT and the associated 5HTP to Trp (5HTP/Trp) and 5HT to Trp (5HT/Trp) ratios. These effects were largely limited by Telotristat (TPH1 inhibitor), and further increased by Phenethylamine (PEA), a TPH1 inducer. In addition, Telotristat was able to limit PEA-induced TPH1 activity.

### Immune Cell Activation

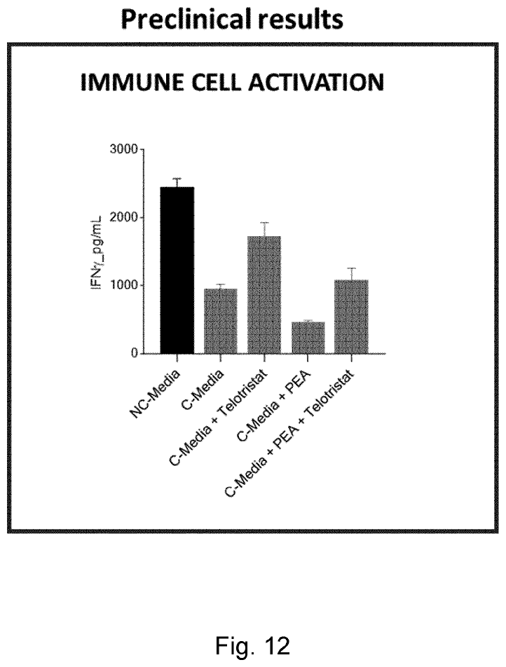

At the end of PBMCs treatment period as described above, cell culture supernatants were collected and subjected to IFN-γ measurement by mean of HTRF (Homogeneous Time Resolved Technology, Perkin Elmer) which serves as a surrogate of immune cell activation (FIG. 12). HTRF is a standard technique well-known in the art and therefore perfectly within the routine of the skilled person.

As shown in FIG. 12, C-Media induced a decrease in anti-CD3 mediated PBMCs activation. This effect was limited by Telotristat and increased by PEA. In addition, PEA-induced TPH1 activation was also associated with a more limited immune cell activation which was alleviated by Teloristat. These data highlight the importance of TPH1 activity on immune cell activation and indicate that TPH1 blockade is necessary to enhance an immune response in a TPH1 high condition.

### REFERENCES

Cianchetta G, Stouch T, Yu W, Shi Z C, Tari L W, Swanson R V, Hunter M J, Hoffman I D, Liu Q. Mechanism of Inhibition of Novel Tryptophan Hydroxylase Inhibitors Revealed by Co-crystal Structures and Kinetic Analysis. Curr Chem Genomics. 2010 Apr. 14; 4:19-26
Qingyun Liu, Qi Yang, Weimei Sun, Pete Vogel, William Heydorn, Xiang-Qing Yu, Zhixiang Hu, Wangsheng Yu, Brandie Jonas, Randy Pineda, Valerie Calderon-Gay, Michael Germann, Emily O'Neill, Robert Brommage, Emily Cullinan, Ken Platt, Alan Wilson, Dave Powell, Arthur Sands, Brian Zambrowicz and Zhi-cai Shi. Journal of Pharmacology and Experimental Therapeutics Apr. 1, 2008, 325 (1) 47-55
Joseph Loscalzo, George Welch, Nitric oxide and its role in the cardiovascular system, Progress in Cardiovascular Diseases, Volume 38, Issue 2, 1995, Pages 87-104, ISSN 0033-0620
Steppan et al., Front. Immunol., 17 Sep. 2013
Pudlo, Marc & Demougeot, Céline & Girard, Corine. (2016). Arginase Inhibitors: A Rational Approach Over One Century: AN EXHAUSTIVE REVIEW OF ARGINASE INHIBITORS. Medicinal Research Reviews. 37.10.1002/med.21419.
Wong V W, Lerner E. Nitric oxide inhibition strategies. Future Sci OA. 2015; 1 (1): FSO35
Wegener G, Volke V. Nitric Oxide Synthase Inhibitors as Antidepressants. Pharmaceuticals (Basel). 2010 Jan. 20; 3 (1): 273-299. #
Sciabola S, Xi H, Cruz D, Cao Q, Lawrence C, Zhang T, Rotstein S, Hughes J D, Caffrey D R, Stanton R V. PFRED: A computational platform for siRNA and antisense oligonucleotides design. PLOS One. 2021 Jan. 22; 16 (1)
Keith T. Gagnon and David R. Corey. Guidelines for Experiments Using Antisense Oligonucleotides and Double-Stranded RNAs. Nucleic Acid Therapeutics. June 2019.116-122.
Magaki S, Hojat S A, Wei B, So A, Yong W H. An Introduction to the Performance of Immunohistochemistry. Methods Mol Biol. 2019; 1897:289-298.
Corchete, L. A., Rojas, E. A., Alonso-López, D. et al. Systematic comparison and assessment of RNA-seq procedures for gene expression quantitative analysis. Sci Rep 10, 19737 (2020).
Holland P M, Abramson R D, Watson R, Gelfand D H. Detection of specific polymerase chain reaction product by utilizing the 5′-3′ exonuclease activity of Thermus aquaticus DNA polymerase. Proc Natl Acad Sci USA. 1991 Aug. 15; 88 (16): 7276-80.
Gaynor E M, Mirsky M L, Lewin H A. Use of flow cytometry and RT-PCR for detecting gene expression by single cells. Biotechniques. 1996 August; 21 (2): 286-91
Mckinnon K M. Flow Cytometry: An Overview. Curr Protoc Immunol. 2018 Feb. 21; 120:5.1.1-5.1.11.
Zhai et al., 2023, Ruminococcus gnavus plays a pathogenic role in diarrhea-predominant irritable bowel syndrome by increasing serotonin biosynthesis, Cell Host & Microbe 31, 33-44, Jan. 11, 2023

What is claimed is:

1. An in vitro method of determining the need of a patient
being diagnosed for,
suffering from or
being at risk of developing
cancer, for a given therapy, the method comprising
a) determining, in vitro, in one or more samples collected from said patient, the activity of at least one enzyme selected from the group consisting of Tryptophan Hydroxylase, 5-HTP decarboxylase, Arginase and/or Nitric oxide synthase (NOS), and
b) determining the patient to be in need of a combined treatment comprising
(i) anti-cancer therapy, and
(ii) an inhibitor of Tryptophan Hydroxylase, 5-HTP decarboxylase, Arginase and/or Nitric oxide synthase (NOS)
if a high activity of the respective enzyme is determined in step a),
c) while determining the patient to be in need of a cancer treatment comprising anti-cancer therapy, if a low activity of the respective enzyme is determined in step a).

2. The in vitro method of claim 1, wherein
the activity of Tryptophan Hydroxylase is determined by measuring the levels of at least a pair of metabolites selected from
a) Tryptophan (Trp) and 5-hydroxythroptophane (5-HTP), and/or
b) Tryptophan (Trp) and 5-hydroxytryptamine (5-HT)
the activity of 5-HTP decarboxylase is determined by measuring the levels of at least a pair of metabolites selected from
c) Tryptophan (Trp) and 5-hydroxytrypütamine (5-HT), and/or
d) 5-hydroxythroptophane (5-HTP) and 5-hydroxytryptamine (5-HT)
the activity of Arginase is determined by measuring the levels of the metabolites Arginine (Arg) and Ornithine (Orn), and/or
the activity of Nitric oxide synthase (NOS) is determined by measuring the levels of the metabolites Arginine (Arg) and Citrulline (Cit).

3. The in vitro method of claim 1 or 2, wherein
the activity of Tryptophan Hydroxylase is determined by calculating the ratio of the levels of
a) 5-hydroxythroptophane (5-HTP) and Tryptophan (Trp), and/or
b) 5-hydroxytryptamine (5-HT) and Tryptophan (Trp),
the activity of 5-HTP decarboxylase is determined by calculating the ratio of the levels of
c) 5-hydroxytryptamine (5-HT) and Tryptophan (Trp) and/or
d) 5-hydroxytryptamine (5-HT) and 5-hydroxythroptophane (5-HTP),
the activity of Arginase is determined by calculating the ratio of the levels of Ornithine (Orn) and Arginine (Arg), and/or
the activity of Nitric oxide synthase (NOS) is determined by calculating the ratio of the levels of Citrulline (Cit) and Arginine (Arg).

3. The in vitro method according to any one of the aforementioned claims, wherein said one or more samples from the patient have been derived from at least one selected from the group consisting of
plasma sample,
serum sample,
urine sample,
saliva sample,
liquor sample, and/or
whole blood sample.

4. The in vitro method according to any one of the aforementioned claims, wherein the level of at least one metabolite is determined by at least one method selected from the group consisting of
Immunoassay
Gas Chromatography/Mass Spectroscopy (GC/MS)
High Performance Liquid Chromatography (HPLC), and/or
Liquid Chromatography/Mass spectroscopy (LC/MS).

5. The in vitro method according to any one of the aforementioned claims, wherein the anti-cancer therapy comprises at least one selected from the group consisting of
chemotherapy
targeted therapy
administration of one or more anti-angiogenic drugs,
administration of one or more TKI (tyrosine kinase inhibitors), and/or
an immunotherapeutic approach.

6. The in vitro method according to claim 5, wherein the immunotherapeutic approach comprises the administration of at least one selected from the group consisting of
immune checkpoint inhibitor,
a bispecific antibody (e.g. BITE) or TCR comprising an immune effector, like a cytokine or an CD3 binding entity
a tumor vaccine
CAR T cell,
TLR agonist.

7. The in vitro method according to claim 6, wherein the immune checkpoint inhibitor is a binder, inhibitor or antagonist of at least one selected from the group consisting of
CTLA-4,
PD-1,
PD-L1,
LAG 3,
TIM3,
OX40,
TIGIT,
ICOS,
41BB (CD137),
VISTA,
and/or GITR.

8. The in vitro method according to claim 7, wherein the binder, inhibitor or antagonist is at least one selected from the group consisting of
Antibody,
Modified antibody format,
Antibody derivative or fragment,
Antibody-based binding protein, and/or
Antibody mimetic.

9. The in vitro method according to any one of claim 6 or 7, wherein the immune checkpoint inhibitor is at least one selected from the group consisting of
Ipilimumab (anti-CTLA-4),
Nivolumab (anti-PD-1),
Pembrolizumab (anti-PD-1),
Cemiplimab (anti-PD-1),
Spartalizumab (anti-PD-1),
Budigalimab (anti-PD1),
Refitanlimab (anti-PD1),
Dostarlimab (anti-PD1)
Atezolizumab (anti-PD-L1),
Avelumab (anti-PD-L1),
Durvalumab (anti-PD-L1),
INCB86550 (anti-PDL1)
Etigilimab (anti-TIGIT),
BGB-A1217 (anti-TIGIT)
BMS-986207 (anti-TIGIT),
AB154 (anti-TIGIT) ASP8374 (anti-TIGIT),
MK 7684 (anti-TIGIT),
Tiragolumab (anti-TIGIT),
INCAGN1949 (anti-OX40),
INCAGN2390 (anti-TIM3),
INCAGN2385 (anti-LAG3),
MCLA-145 (PDL1×CD137), and/or
MEDI5752 (PD1×CTLA4)

10. The in vitro method according to any one of the aforementioned claims, wherein
a) the inhibitor of Tryptophan Hydroxylase is at least one selected from the group consisting of
Fenclonine
LP-533401
LP-533401 hydrochloride
LP-521834
LP-534193
LP-615819
Telotristat ethyl
Telotristat etiprate
Telotristat
6-Fluorotryptophan
p-Ethynylphenylalanine (4-Ethynyl-L-phenylalanine)·
p-Ethynylphenylalanine hydrochloride (4-Ethynyl-L-phenylalanine hydrochloride)
LX-1031
PCPA methyl ester hydrochloride
Rodatristat
Rodatristat ethyl
ACT-678689 and/or
one or more antisense oligonucleotides.
b) the inhibitor of 5-HTP decarboxylase is at least one selected from the group consisting of
Ro 4602, and/or
one or more antisense oligonucleotides.
c) the inhibitor of Arginase is at least one selected from the group consisting of
Resveratrol
Norvaline
N-hydroxy-l-arginine
N-hydroxy-nor-L-arginine (nor-NOHA)
N-hydroxy-guanidinium
2 (S)-amino-6-boronohexanoic acid (ABH)
S-(2-boronoethyl)-L-cysteine (S-2-BEC),
(R)-2-amino-6-borono-2-(2-(piperidin-1-yl)ethyl) hexanoic acid, and/or
one or more antisense oligonucleotides, and/or.
d) the inhibitor of Nitric oxide synthase is at least one selected from the group consisting of:
L-NANA (L-NG-Methyl-L-arginine)
N-PLA (L-NG-Propyl-L-arginine)
L-NNA (L-NG-Nitroarginine)
L-NAME (L-NG-Nitroarginine methyl ester)
L-NAA NG-Amino-L-arginine
ADMA (NG,NG-Dimethyl-L-arginine)
SDMA (NG,NG′-Dimethyl-L-arginine)
L-NIL (L-N6-(1-Imino-ethyl) lysine)
L-Thiocitrulline
S-Methyl-L-Thiocitrulline
Agmatine (1-Amino-4-guanidinobutane)
L-Canavanine
L-NMMA (Tilarginine)
N(G)-methyl-l-arginine hydrochloride (546C88)
Ng-nitro-l-arginine (L-NArg)
AMIDINES
L-NIO N8-(Iminoethyl)-L-ornithine
Ethyl-L-NIO
Vinyl-L-NIO
1400W (N-(3-(Aminomethyl)benzyl) acetamidine)
INDAZOLE DERIVATES
7-NI (7-Nitroindazole)
7-NI-Br (7-Bromonitroindazole)
Imidazole derivates
TRIM (1-[2-(Trifluoromethyl)phenyl-imidazole
2-IMINOPIPERIDINE DERIVATES
2-Imino-4-methylpiperidine
HYDRAZINE DERIVATES
Aminoguanidine
ISOTHIOUREAS
S-(2-Aminoethyl) isothiourea
1,3-PBIT (S,S′-(1,3-Phenylene-bis(1,2-ethanediyl))bis-isothiourea)
1,4-PBIT (S,S′-(1,4-Phenylene-bis(1,2-ethanediyl))bis-isothiourea)
α-Guanidinoglutaric Acid,
Methylene blue,
ODQ ([1H-[1,2,4]Oxadiazole [4,3-a]quinoxalin-1-one], and/or
one or more antisense oligonucleotides.

11. The in vitro method according to any one of the aforementioned claims, wherein the inhibitor of Tryptophan Hydroxylase is Telotristat ethyl, Telotristat etiprate, or Telotristat.

12. The in vitro method according to any one of the aforementioned claims, wherein the inhibitor of Nitric oxide synthase is L-NMMA (Tilarginine).

13. A kit for carrying out a method according to any one of the aforementioned claims, said kit comprising means for detecting at least one metabolite selected from the group consisting of Arginine, Tryptophan, 5-HT, 5-HTP, Citrulline and/or Ornithine in an immunoassay.

14. A drug combination (in the manufacture of one or more medicaments) for use in the treatment of a patient being diagnosed for, suffering from or being at risk of developing cancer, said patient being characterized as having, as determined in one or more samples collected from said patient, a high activity of at least one of Tryptophan Hydroxylase, 5-HTP decarboxylase, Arginase and/or Nitric oxide synthase, which drug combination comprises
(i) anti-cancer therapy, and
(ii) an inhibitor of Tryptophan Hydroxylase, 5-HTP decarboxylase, Arginase and/or Nitric oxide synthase
